(19) United States
(12) Patent Application Publication
(10) Pub. No.: US 2010/0247435 A1
(43) Pub. Date: Sep. 30, 2010

(54) MEASUREMENT OF NEURAL ACTIVITY

(76) Inventor: Erik Arstad, London (GB)

Correspondence Address: GE HEALTHCARE, INC., IP DEPARTMENT 101 CARNEGIE CENTER, PRINCETON, NJ, 08540-6231, US

(21) Appl. No.: 12/663,708

(22) PCT Filed: Jun. 12, 2008
(86) PCT No.: PCT/EP08/57418

§ 371 Date: May 26, 2010

Related U.S. Application Data
(60) Provisional application No. 60/943,872, filed on Jun. 14, 2007.

(51) Int. Cl. A61K 51/04; A61K 49/00; C07D 231/12; A61P 43/00

(52) U.S. Cl. 424/1.89; 424/9.1; 548/374.1

(57) ABSTRACT

The present invention provides a method for determination of neural activity in a sample. The method of the invention is useful for the determination of neural activity in both in vivo and in vitro samples and is particularly useful in providing diagnostic information in subjects suspected to have a neurological condition that leads to disturbed neurological signalling. Also provided by the invention are compounds suitable for use in the method of the invention, and a pharmaceutical composition useful for carrying out the method of the invention.

### TECHNICAL FIELD OF THE INVENTION

The present invention relates to measurement of neural activity. In particular the invention relates to a method of measuring neural activity using a compound labelled with a detectable label which can target activated neural cells. The method finds use in the diagnosis of conditions associated with disturbed neural signalling.

### DESCRIPTION OF RELATED ART

Neural activity is mediated by voltage gated sodium channels (VGSC), which transmit activation potentials by allowing an influx of sodium ions when triggered by a change in the membrane potential. Once activated, VGSC rapidly (within around 1 millisecond) close and go into a prolonged inactivated state, before returning to their resting state (closed but activatable). Compounds are known in the art that selectively target the inactivated state.

Shao et al [2004 J. Med. Chem. 47 pp 4277-4285] disclose phenoxyphenyl pyridine compounds which are potent state-dependent VGSC blockers. Two of the compounds were tested in the Chung in vivo rat model of painful neuropathy and it was demonstrated that the compounds reversed tactile allodynia in a dose-dependent manner. These compounds were therefore concluded to have potential in the treatment of neuropathic pain. Yang et al [2004 J. Med. Chem. 47 pp 1547-1552] disclose a series of 3-(4-phenoxyphenyl)pyrrazoles which are also potent state-dependent VGSC blockers. One of the compounds was tested in the Chung model. It was shown that the compound had antiallodynic effects that compared favourably with carbamezepine, an anticonvulsant agent currently used in the treatment of neuropathic pain. Liberatore et al [Bioorg. Med. Chem. Lett. 2004 14 pp 3521-3523] disclose a series of 2-alkyl-4-arylimidazoles that display nanomolar IC₅₀ values for their ability to inhibit binding of tritiated batrachotoxin to the rat brain site 2 sodium channel. The sodium channel binding properties of these compounds were shown by Liberatore et al to compare favourably with known drugs used for neurological disorders such as lidocaine, carbamazepine and lamotrigine.

Several techniques have been developed to image the operational organization of the human brain, of which ¹⁸FDG, blood flow tracers and fMRI are extensively used. However, none of these methods measure neural activity directly, but instead targets secondary effects such as energy/oxygen consumption and changes to local blood flux. As a result, current methods suffer from low accuracy, frequent overestimation of incoming input and local processing over spiking activity, and fail to reveal the neural pathway between activated regions (N K Logothetis, Nature, 412, 2001, pp 150-157).

There is consequently a need for improvements in the determination of neural activity.

### SUMMARY OF THE INVENTION

The present invention provides a method for determination of neural activity in a sample. The method of the invention is useful for the determination of neural activity in both in vivo and in vitro samples and is particularly useful in providing diagnostic information in subjects suspected to have a condition that is associated with disturbed neural signalling. Also provided by the invention are compounds suitable for use in the method of the invention, and a pharmaceutical composition useful for carrying out the method of the invention.

### DETAILED DESCRIPTION OF THE INVENTION

### Method of Determining Neural Activity

In one aspect the invention relates to a method of determining neural activity in a sample comprising detection of signals emitted by a labelled compound present in said sample, characterised in that said labelled compound has selective affinity for the inactivated or active state of voltage-gated sodium channels (VGSC).

The method of the invention comprises the following steps:
(i) contacting the labelled compound with the sample to allow the labelled compound to bind to VGSC in the sample that are in the inactivated or active state;
(ii) detecting signals emitted by the labelled compound;
(iii) converting said signals into numerical data or an image

While the method of the invention has application for evaluation of normal physiology, it is preferably applied in the diagnosis of a neurological condition that is associated with disturbed neural signalling. Examples of such conditions include (but are not necessarily limited to) epilepsy, pathological pain, multiple sclerosis, Parkinson's disease, Alzheimers, schizophrenia, and depression.

### Types of Sample

The term “sample” is intended to cover human and animal samples in vitro, ex vivo, and in vivo.

An “in vitro sample” is a tissue or a fluid sample taken from a human or an animal body and analysed outside the body. The step of contacting the labelled compound to the sample is carried out by bringing both together in a suitable medium, such as a physiological buffer solution. Preferred in vitro samples for use in the method of the invention are tissue samples taken from the central or peripheral nervous systems, or fluid samples such as blood, serum, plasma, or cerebrospinal fluid. Most preferred in vitro samples are fluid samples. Where the method is carried out on an in vitro sample, the labelled compound suitably comprises a detectable label which is a reporter suitable for in vitro diagnostic methods. Such detectable labels are outlined in more detail later. Means of detecting signals emitted by such labelled compounds are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the coloured label. The signals detected are representative of the number of activated VGSCs in the sample in question.

The term ex vivo refers to a biological process or reaction taking place outside of a living cell or organism. A typical “ex vivo sample” is a cell culture, with preferred cell cultures for use in the method of the invention being derived from cells of the central or peripheral nervous systems. The detectable labels used when the method of the invention is carried out on an ex vivo sample are similar to those used for an in vitro sample.

An “in vivo sample” is one which is present in a living human or animal subject, and for the purposes of the present invention is typically an organ or organ system. Preferably, the in vivo sample is part of the nervous system of a subject, and is most preferably the brain. When the sample is an in vivo sample, the contacting step may be carried out by administration of the labelled compound to the human or animal subject to ensure that it comes into contact with cells in the sample that may have an increased expression of VGSCs. Administration is preferably achieved intravenously.

The method of the invention is preferably carried out on an in vivo sample, which is most preferably a human subject, or an organ or organ system in said human subject.

Where the method of the invention is carried out in vivo, the signals emitted by the labelled compound are preferably converted into an image. This may be achieved by an in vivo imaging technique, e.g. single-photon emission computed tomography (SPECT), positron-emission tomography (PET), magnetic resonance imaging (MRI), or optical imaging. Preferred in vivo imaging techniques are SPECT and PET, most preferably PET.

Preferably, labelled compounds for use in the method of the invention do not undergo facile metabolism in vivo, and hence most preferably exhibit a half-life in vivo of 60 to 240 minutes in humans. The labelled compound is preferably excreted via the kidney (i.e. exhibits urinary excretion), preferably exhibiting a signal-to-background ratio at diseased foci of at least 1.5, most preferably at least 5, with at least 10 being especially preferred. Where the labelled compound comprises a radioisotope, clearance of one half of the peak level of labelled compound which is either non-specifically bound or free in vivo, preferably occurs over a time period less than or equal to the radioactive decay half-life of the radioisotope.

The effective in vivo dose of a labelled compound, or a salt thereof, will vary depending on the exact labelled compound to be administered, the weight of the patient, and other variables as would be apparent to a physician skilled in the art. Generally, the dose would lie in the range 0.001 μg/kg to 10 μg/kg, preferably 0.01 μg/kg to 1.0 μg/kg.

In an alternative embodiment, the method of the invention provides for a method of in vivo imaging of neural activity in a subject wherein said subject is previously administered with the pharmaceutical composition of the invention. By “previously administered” is meant that the step involving the clinician, wherein the imaging agent is given to the patient e.g., intravenous injection, has already been carried out.

The method of the invention may also be applied for carrying out a method for monitoring the effect of treatment of a subject with a drug to combat a neurological condition associated with disturbed neural signalling, said method comprising administering to said subject the radiopharmaceutical composition of the invention and detecting the uptake of said labelled compound, said administration and detection optionally but preferably being effected before, during and after treatment with said drug.

In another aspect, the invention provides the labelled compound of the invention for use in the method of the invention.

In a further aspect, the invention provides for the use of the labelled compound in the manufacture of a pharmaceutical composition for use in the method of the invention.

### Detectable Labels

In the method of the invention, the labelled compound having selective affinity for the inactivated or active state of voltage-gated sodium channels (VGSC) comprises a detectable label selected from:
(i) a gamma-emitting radioactive halogen;
(ii) a positron-emitting radioactive non-metal;
(iii) a hyperpolarised NMR-active nucleus;
(iv) a beta-emitter suitable for intravascular detection;
(v) a reporter suitable for in vivo optical imaging;
(vi) a reporter suitable for in vitro diagnostic methods;
(vii) a radioactive metal ion; and,
(viii) a paramagnetic metal ion.

When the detectable label is a “gamma-emitting radioactive halogen”, the radiohalogen is suitably chosen from ¹²³I, ¹³¹I, ¹²⁵I or ⁷⁷Br. A preferred gamma-emitting radioactive halogen is ¹²³I.

When the detectable label is a “positron-emitting radioactive non-metal”, suitable such positron emitters include: ¹¹C, ¹³N, ¹⁵O, ¹⁷F, ¹⁸F, ⁷⁵Br, ⁷⁸Br or ¹²⁴I. Preferred positron-emitting radioactive non-metals are ¹¹C, ¹³N, ¹⁸F and ¹²⁴I, especially ¹¹C and ¹⁸F.

When the detectable label is a “hyperpolarised NMR-active nucleus”, such NMR-active nuclei have a non-zero nuclear spin, and include ¹³C, ¹⁵N, ¹⁹F, ²⁹Si and ³¹P. Of these, ¹³C is preferred.

When the detectable label is a “beta-emitter suitable for intravascular detection”, suitable such beta-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br. ³⁸Cl, ³⁹Cl, ⁸²Br and ⁸³Br are preferred.

When the detectable label is a “reporter suitable for in vivo optical imaging”, it is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy 3B, Cy3.5, Cy5, Cy5.5, Cy7, Cy7.5, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared (NIR) region, between 400 nm and 3 μm, particularly between 600 and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

A “reporter suitable for in vitro diagnostic methods” is a label detectable by spectroscopic, photochemical, biochemical, immunochemical, electrical, optical or chemical means. Useful such labels in the context of the present invention include magnetic beads (e.g. DYNABEADS™), fluorescent dyes (e.g., fluorescein isothiocyanate, Texas red, rhodarmine, green fluorescent protein), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g. polystyrene, polypropylene, latex, etc.) beads. Of these, radiolabels are preferred, in particular ³H, ¹²⁵I and ¹⁴C.

When the imaging moiety is a “radioactive metal ion”, i.e. a radiometal, suitable radiometals can be either positron emitters such as ⁶⁴Cu, ⁴⁸V, ⁵²Fe, ⁵⁵Co, ^{94m}Tc or ⁶⁸Ga; gamma-emitters such as ^{99m}Tc, ¹¹¹In, ¹¹³In, or ⁶⁷Ga. Preferred radiometals are ^{99m}Tc, ⁶⁴Cu, ⁶⁸Ga and ¹¹¹In. Most preferred radiometals are gamma-emitters, especially ^{99m}Tc.

When the imaging moiety is a “paramagnetic metal ion”, suitable such metal ions include: Gd(III), Mn(II), Cu(II), Cal), Fe(III), Co(II), Er(II), Ni(II), Eu(III) or Dy(III). Preferred paramagnetic metal ions are Gd(III), Mn(II) and Fe(III), with Gd(III) being especially preferred.

Preferred detectable labels are those which can be detected externally in a non-invasive manner following administration in vivo such as by means of SPECT, PET and MR, preferably SPECT and PET. Most preferred detectable labels are radioactive, in particular (i), (ii) and (vii) from the list of detectable labels above, and especially preferably (i) and (ii) from this list. Of these, ¹²³I, ¹⁸F and ¹¹C are preferred.

### Labelled Compounds

The method of the invention is preferably carried out using a particular labelled compound, which in turn forms another aspect of the invention. Particular labelled compounds are now described in more detail.

The term “labelled compound” is used herein to mean a labelled compound per se, or a salt or solvate thereof.

Suitable salts according to the invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

Suitable solvates according to the invention include those formed with ethanol, water, saline, physiological buffer and glycol.

“Selective affinity” for inactivated or active state of VGSC means that the labelled compound has greater binding potential for the inactivated or active state as compared with the resting state. Such selective affinity may be measured for example by measuring the dissociation constant for binding to the resting state of rNaᵥ1.2 channels stably expressed in HEK-293 cells (Yang et al, J. Med. Chem. 2004 47 pp 1547-1552).

Preferably, the Kᵢ of the labelled compound for the inactivated or activated state is between 1 nM and 100 nM, most preferably between 1 nM and 50 nM and most especially preferably between 1nM and 30 nM.

A particular labelled compound of the invention is a compound of Formula I:

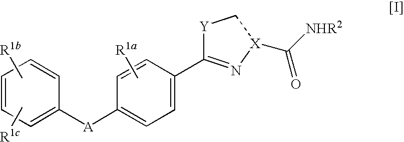

labelled with a detectable label; and wherein:

R^{1a} to R^{1c} are independently an R¹ group selected from hydrogen, C₁₋₃ alkyl, C₁₋₃ alkoxy, hydroxyl, C₁₋₃ hydroxyalkyl, thiol, C₁₋₃ thioalkyl, C₁₋₃ thioalkoxy, halo, C₁₋₃ haloalkyl, C₁₋₃ haloalkoxy, nitro, C₁₋₃ nitroalkyl, C₁₋₃ nitroalkoxy, C₄₋₆ cycloalkyl, or a C₃₋₅ heterocycloalkyl group attached via a C₁₋₃ alkyl;

R² is hydrogen, C₁₋₆ alkyl, C₁₋₆ haloalkyl, or a C₄₋₆ cycloalkyl group attached via a C₁₋₆ alkyl;

A is S or O;

X is C and the dotted bond is a double bond, or X is N and the dotted bond is a single bond; and,

Y is CH₂ or CH═CH.

The term “labelled with a detectable label” means that either (i) the isotopic version of an atom intrinsic to Formula I is a detectable label or (ii) a chemical group comprising a detectable label is conjugated to a compound of Formula I.

Unless otherwise specified, the term “alkyl” alone or in combination, means a straight-chain or branched-chain alkyl radical containing preferably from 1 to 10 carbon atoms, more preferably from 1 to 5 carbon atoms, most preferably 1 to 3 carbon atoms. Examples of such radicals include, but are not limited to, methyl, ethyl, n-propyl, isopropyl, n-butyl, isobutyl, sec-butyl, tert-butyl, pentyl, iso-amyl, hexyl, octyl.

Unless otherwise specified, the term “hydroxyalkyl”, alone or in combination, means a alkyl radical as defined above wherein at least one hydrogen atom has been replaced by a hydroxyl group, but no more than one hydrogen atom per carbon atom; preferably, 1 to 4 hydrogen atoms have been replaced by hydroxyl groups; more preferably, 1 to 2 hydrogen atoms have been replaced by hydroxyl groups; and most preferably, one hydrogen atom has been replaced by a hydroxyl group.

Unless otherwise specified, the term “alkoxy”, alone or in combination, means an alkyl ether radical wherein the term alkyl is as defined above. Examples of suitable alkyl ether radicals include, but are not limited to, methoxy, ethoxy, n-propoxy, isopropoxy, n-butoxy, iso-butoxy, sec-butoxy, tert-butoxy.

Unless otherwise specified, the term “cycloalkyl”, alone or in combination, means a saturated or partially saturated monocyclic, bicyclic or tricyclic alkyl radical wherein each cyclic moiety contains preferably from 3 to 8 carbon atom ring members, more preferably from 3 to 7 carbon atom ring members, most preferably from 4 to 6 carbon atom ring members, and which may optionally be a benzo fused ring system which is optionally substituted as defined herein with respect to the definition of aryl. Examples of such cycloalkyl radicals include, but are not limited to, cyclopropyl, cyclobutyl, cyclopentyl, cyclohexyl, cycloheptyl, octahydronaphthyl, 2,3-dihydro-1H-indenyl, adamantyl.

The term “halo” means a substituent selected from fluorine, chlorine, bromine or iodine. “Haloalkyl” and “haloalkoxy” are alkyl and alkoxy groups, respectively, as defined above substituted with one or more halo groups.

The term “thiol” means an —SH group. “Thioalkyl” and “thioalkoxy” are —SR groups wherein R is an alkyl or an alkoxy, respectively, as defined above.

The term “nitro” means an —NO₂ group. “Nitroalkyl” and “nitroalkoxy” are alkyl and alkoxy groups, respectively, as defined above, substituted with an —NO₂ group.

Preferably, for compounds of Formula I:
R¹ is hydrogen, methyl, methoxy, thiol, thiomethyl, thiomethoxy, halo, halomethyl, halomethoxy, nitro, nitromethyl, or nitromethoxy;
R² is hydrogen or C₁₋₆ haloalkyl;
A is O;
X is N and the dotted bond is a single bond; and,
Y is CH₂.

Most preferably, for compounds of Formula I:
R¹ is hydrogen, methyl or halo;
R² is hydrogen;
A is O;
X is N and the dotted bond is a single bond; and,
Y is CH₂.

In an alternative, for preferred compounds of Formula I:
R¹ is hydrogen, methyl, methoxy, thiol, thiomethyl, thiomethoxy, halo, halomethyl, halomethoxy, nitro, nitromethyl, or nitromethoxy;
R² is hydrogen, or a C₄₋₆ cycloalkyl group or C₃₋₅ heterocycloalkyl group attached via a C₁₋₃ alkyl;
A is O;
X is C and the dotted bond is a double bond; and,
Y is CH═CH.

For these alternative preferred compounds of Formula I, it is most preferred that:
R¹ is hydrogen, methyl or halo;
R² is hydrogen, or C₃₋₅ heterocycloalkyl group attached via a C₁₋₃ alkyl;
A is O;
X is C and the dotted bond is a double bond; and,
Y is CH═CH.

Most preferably for both alternative preferred labelled compounds of Formula I described above, one of R^{1a}, R^{1b}, R^{1c}, or —C═O—NHR² comprises the detectable label. When the detectable label is ¹²³I or ¹⁸F it is preferably comprised in one of R^{1a}, R^{1b} or R^{1c}, and most preferably in one of R^{1b} or R^{1c}. When one of ¹²³I or ¹⁸F is comprised in either of R^{1b} or R^{1c}, it is preferably at the 3-, 4- or 5-position relative to the oxygen bridge. When the detectable label is ¹¹C, it is preferably the carbonyl carbon of the —C═O—NHR² group.

Some examples of these most preferred compounds of Formula I labelled with a detectable label are as follows:

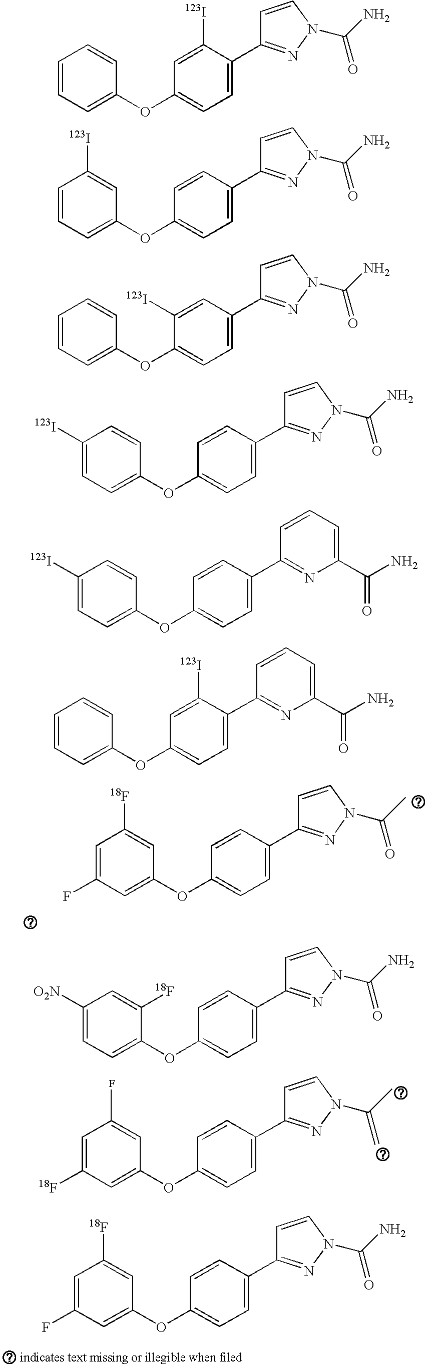

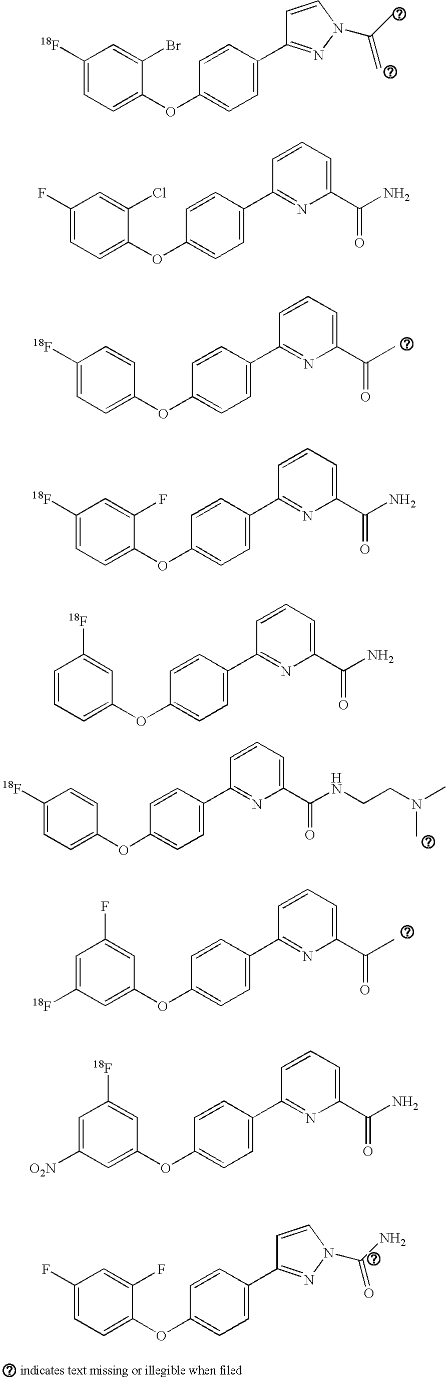

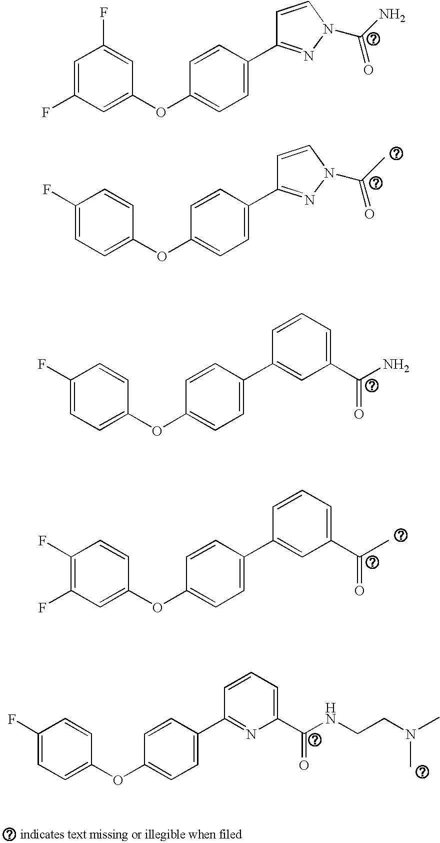

In another embodiment, the labelled compound of the invention is a compound of Formula II:

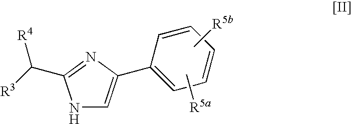

labelled with a detectable label and wherein:
R³ and R⁴ are independently selected from hydrogen, C₁₋₁₀ alkyl, C₁₋₁₀ alkoxy, C₁₋₁₀ alkoxyalkyl, C₁₋₄ haloalkyl, C₁₋₄ haloalkenyl, C₁₋₃ haloalkoxy; C₄₋₆ cycloalkyl, C₁₋₂₀ PEGalkyl or C₁₋₂₀ PEG; and,
R^{5a} and R^{5b} are independently an R⁵ group selected from hydrogen, C₁₋₄ alkyl, halo, C₁₋₃ haloalkenyl, C₁₋₃ alkoxy, or is a 5- or 6-membered aromatic ring system having 0-3 heteroatoms selected from N, S and O and optionally substituted with C₁₋₃ alkyl, halo, or C₁₋₃ haloalkyl.

The term “PEG” refers to a chain comprising polyethylene glycol units alone, and the term “PEGalkyl” refers to a chain comprising alkyl and polyethylene glycol units. A polyethylene glycol unit has the structure —(CH₂)₂—O—.

Preferably, for Formula II:
R³ is C₁₋₆ alkyl, C₁₋₆ alkoxy, C₁₋₄ haloalkenyl, C₁₋₄ haloalkyl or C₁₋₃ haloalkoxy;
R⁴ is C₁₋₆ alkyl, C₁₋₆ alkoxy; and,
R⁵ is hydrogen, iodo, 2-iodo-C₁₋₃ alkenyl, methoxymethyl, phenyl, 4-fluorophenyl, 4-iodophenyl, pyridyl, 2-fluoroethyl-1,2,3-triazole.

Most preferably, for Formula II:
R³ is propyl, methoxyethyl, iodopropenyl, fluoropropyl or fluoroethoxy;
R⁴ is propyl or methoxyethyl; and,
R⁵ is hydrogen, iodine, 2-iodoalkenyl, methoxymethane, phenyl, 4-fluorophenyl, 4-iodophenyl, pyridine, or 2-fluoroethyl-1,2,3-triazole.

Most preferably for Formula II, one of R³, R^{5a}, or R^{5b} comprises the detectable label. When the detectable label is either ¹²³I or ¹⁸F, it is preferably comprised in R³ or in R^{5b} at the 4-position of the phenyl ring. When the detectable label is ¹¹C or ^{99m}Tc, it is preferably comprised in R^{5b} at the 4-position of the phenyl ring. Examples of certain preferred labelled compounds of Formula II are as follows:

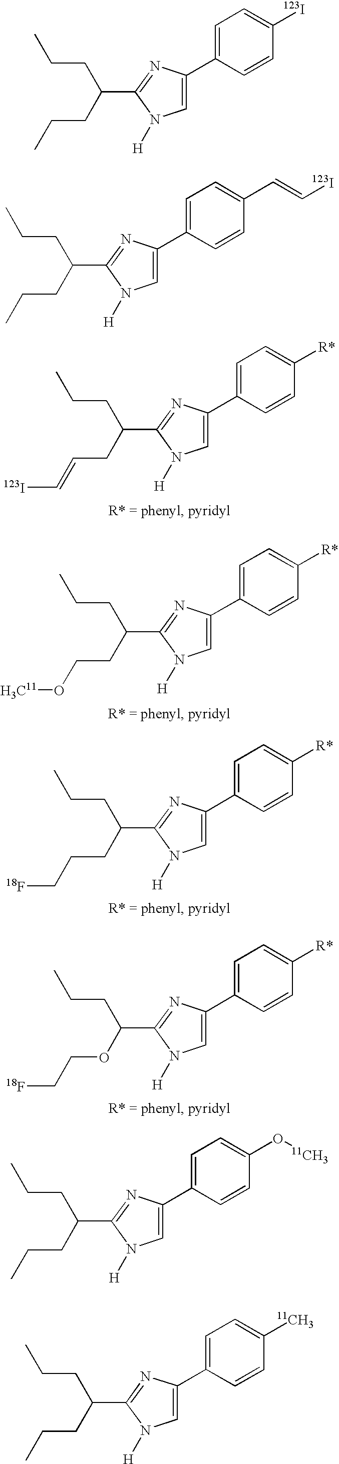

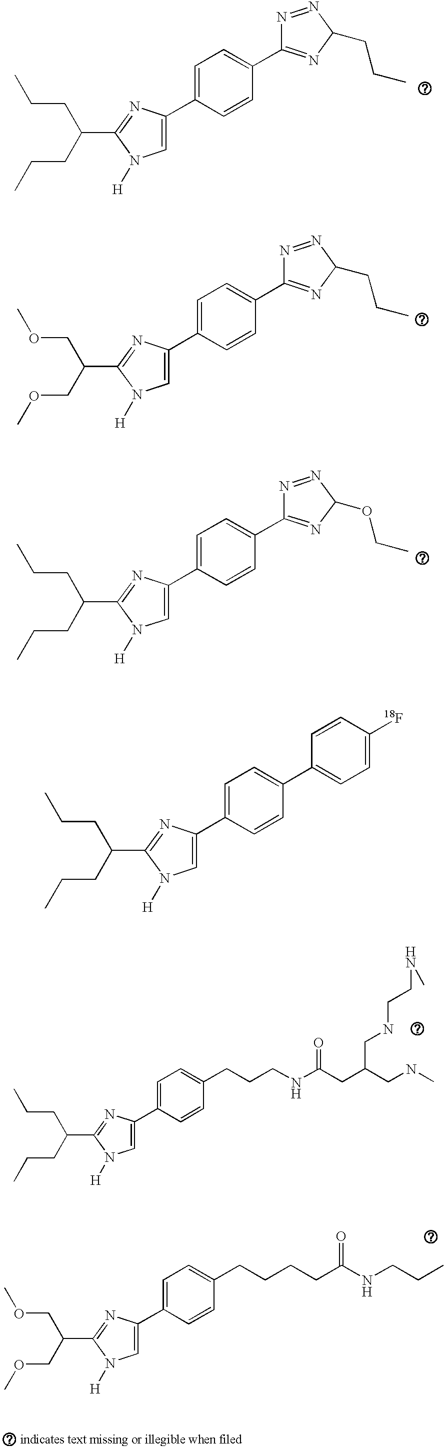

### Preparation of Labelled Compounds

Synthetic routes for various unlabelled compounds of Formula I are described by Shao [J. Med. Chem. 2004 47 pp 4277-4285] and by Yang [J. Med. Chem. 2004 47 pp 1547-1552]. Synthetic routes for various unlabelled compounds of Formula II are described by Liberatore [Bioorg. Med. Chem. Lett. 2004 14 pp 3521-3523].

Labelled compounds of Formulas I and II may be conveniently prepared by reaction of a precursor compound with a suitable source of the desired detectable label.

A “precursor compound” comprises a derivative of a labelled compound, designed so that chemical reaction with a convenient chemical form of the detectable label occurs site-specifically; can be conducted in the minimum number of steps (ideally a single step); and without the need for significant purification (ideally no further purification), to give the desired imaging agent. Such precursor compounds are synthetic and can conveniently be obtained in good chemical purity. The precursor compound may optionally comprise a protecting group for certain functional groups of the precursor compound.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Methods for obtaining certain labelled compounds of the invention are now provided.

### Radioiodination

Where the detectable label is radioiodine, suitable precursor compounds are those which comprise a derivative which either undergoes electrophilic or nucleophilic iodination or undergoes condensation with a labelled aldehyde or ketone. Examples of the first category are:
(a) organometallic derivatives such as a trialkylstannane (eg. trimethylstannyl or tributylstannyl), or a trialkylsilane (eg. trimethylsilyl) or an organoboron compound (eg. boronate esters or organotrifluoroborates);
(b) a non-radioactive alkyl bromide for halogen exchange or alkyl tosylate, mesylate or triflate for nucleophilic iodination;
(c) aromatic rings activated towards electrophilic iodination (e.g. phenols) and aromatic rings activated towards nucleophilic iodination (e.g. aryl iodonium salt aryl diazonium, aryl trialkylammonium salts or nitroaryl derivatives).

The precursor compound for radioiodination preferably comprises: a non-radioactive halogen atom such as an aryl iodide or bromide (to permit radioiodine exchange); an activated aryl ring (e.g. a phenol group); an organometallic substituent (e.g. trialkyltin, trialkylsilyl or organoboron compound); or an organic substituent such as triazenes or a good leaving group for nucleophilic substitution such as an iodonium salt. Preferably for radioiodination, the precursor compound comprises an organometallic substituent, most preferably trialkyltin.

Precursor compounds and methods of introducing radioiodine into organic molecules are described by Bolton [J. Lab. Comp. Radiopharm., 45, 485-528 (2002)]. Suitable boronate ester organoboron compounds and their preparation are described by Kabalaka et al [Nucl. Med. Biol., 29, 841-843 (2002) and 30, 369-373 (2003)]. Suitable organotrifluoroborates and their preparation are described by Kabalaka et al [Nucl. Med. Biol., 31, 935-938 (2004)].

Examples of aryl groups to which radioactive iodine can be attached are given below:

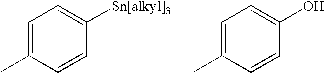

wherein alkyl in this case is preferably methyl or butyl. These groups contain substituents which permit facile radioiodine substitution onto the aromatic ring. Alternative substituents containing radioactive iodine can be synthesised by direct iodination via radiohalogen exchange, e.g.

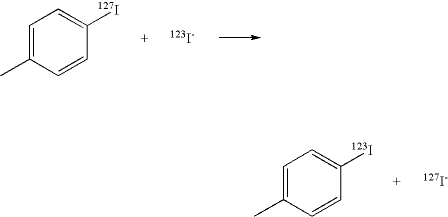

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

For labelled compounds of Formula I, said precursor compound for radioiodination is of Formula Ia:

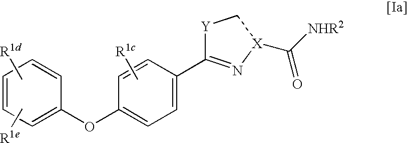

wherein:
one of R^{1c}-R^{1e} is non-radioactive iodine, hydroxyl, or is [C₁₋₆alkyl]₃Sn—Z— wherein Z can be a bond, C₁₋₆ alkyl, or C₁₋₆ alkenyl, and the remaining two are independently an R¹ group as defined for Formula I;
R² is as defined for Formula I; and,
X and Y are as defined for Formula I.

Examples of precursor compounds of Formula Ia for radioiodination are:

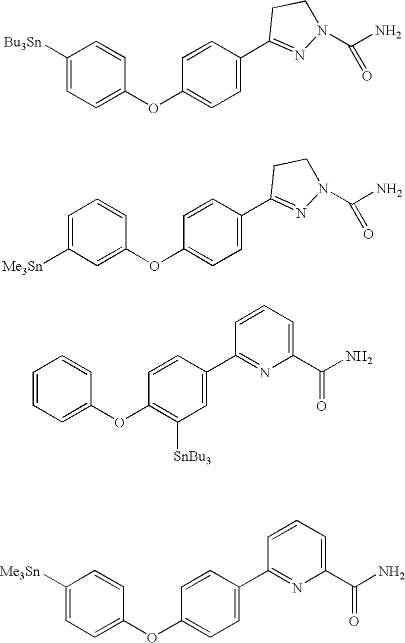

For labelled compounds of Formula II, said precursor compound for radioiodination is of Formula IIa:

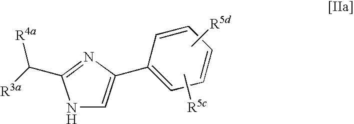

wherein:

one of R^{3a} or R^{4a} is [C₁₋₆alkyl]₃Sn—Z— wherein Z can be a bond, C₁₋₆ alkyl, or C₁₋₆ alkenyl, or one of R^{5c} or R^{5d} is non-radioactive iodine, hydroxyl, or is [C₁₋₆alkyl]₃Sn—Z— wherein Z can be a bond, C₁₋₆ alkyl, or C₁₋₆ alkenyl, and for the remaining groups:

R^{3a} is R³ as defined above for Formula II;

R^{4a} is R⁴ as defined above for Formula II; and,

R^{5c} and R^{5d} are independently an R⁵ group as defined above for Formula II.

Examples of precursor compounds of Formula IIa for radioiodination are:

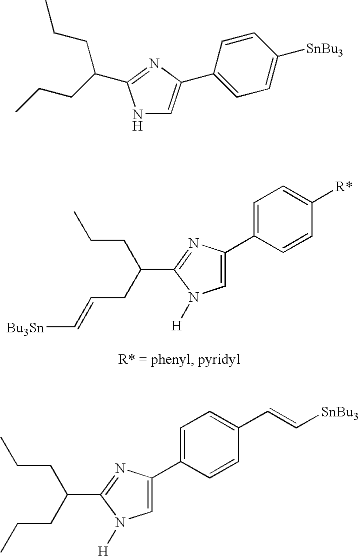

The trialkyltin precursor compounds above are made from the non-radioactive version of the radioiodine compound via a palladium reaction with [alkyl]₃SnSn[alkyl]₃. The reaction as it takes place at the substituent is as follows:

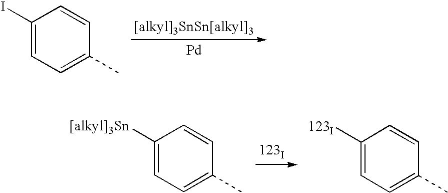

### Radiofluorination

When the detectable label is a radioactive isotope of fluorine the radiofluorine atom may form part of a fluoroalkyl or fluoroalkoxy group, since alkyl fluorides are resistant to in vivo metabolism. Fluoroalkylation may be carried out by reaction of a precursor compound containing a reactive group such as phenol, thiol and amide with a fluoroalkyl group. ¹⁸F can also be introduced by alkylation of N-haloacetyl groups with a ¹⁸F(CH₂)₃OH reactant, to give —NH(CO)CH₂O(CH₂)₃¹⁸F derivatives.

Alternatively, the radiofluorine atom may be attached via a direct covalent bond to an aromatic ring such as a benzene ring. For such aryl systems, ¹⁸F-fluoride nucleophilic displacement from an aryl diazonium salt, aryl nitro compound or an aryl quaternary ammonium salt are suitable routes to aryl-¹⁸F derivatives.

Radiofluorination may be carried out via direct labelling using the reaction of ¹⁸F-fluoride with a suitable chemical group in the precursor compound having a good leaving group, such as an alkyl bromide, alkyl mesylate or alkyl tosylate.

A ¹⁸F-labelled compound may be obtained by formation of ¹⁸F fluorodialkylamines and subsequent amide formation when the ¹⁸F fluorodialkylamine is reacted with a precursor compound containing, e.g. chlorine, P(O)Ph₃ or an activated ester.

For labelled compounds containing a triazole, a further method for the introduction of ¹⁸F is to react a precursor compound comprising an alkyne or azide substituent with [¹⁸F]fluoroalkylazide or [¹⁸F]fluoroalkyne, respectively. This labelling strategy is described in detail in WO 2006/067376.

Further details of synthetic routes to ¹⁸F-labelled derivatives are described by Bolton, J. Lab. Comp. Radiopharm., 45, 485-528 (2002).

For labelled compounds of Formula I, said precursor compound for radiofluorination is of Formula Ib:

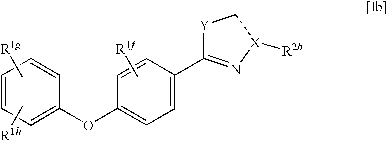

wherein:
one of R^{1f}, R^{1g}, R^{1h} or R^{2b} is azide, C₁₋₆ terminal alkyne, hydroxyl, an N-haloacetyl; or is a reactive group such as phenol, thiol, or amide; or comprises a leaving group such as nitro, trimethylammonium, alkyl bromide, alkyl mesylate, or alkyl tosylate;
and wherein for the remaining groups:
R^{1f}-R^{1h} are independently an R¹ group as defined above for Formula I;
R^{2b} is R² as defined above for Formula I; and,
X and Y are as defined above for Formula I.

For preferred precursors of Formula Ib:
one of R^{1g} and R^{1h} is nitro or trimethylammonium and the other is fluorine, chlorine, nitro or bromine, and for the remaining groups:
R^{1f}-R^{1h} are independently an R¹ group as defined above for Formula I; and,
R^{2b} is R² as defined above for Formula I.

The nitro or trimethylammonium group acts as a leaving group (LG) which can be substituted with ¹⁸F—, and the fluorine, chlorine, nitro or bromine group acts as an electron-withdrawing group (EWG), e.g.:

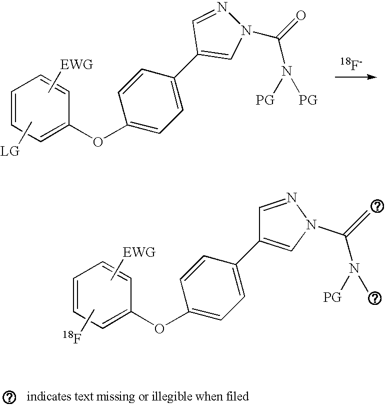

wherein PG is a protecting group as defined above.

Examples of precursor compounds of Formula Ib are:

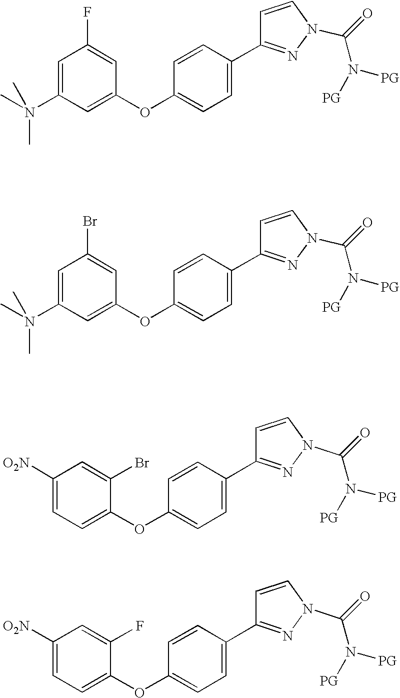

For labelled compounds of Formula II, said precursor compound for radiofluorination is of Formula IIb:

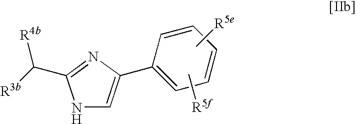

wherein either one of R^{3b} and R^{4b} comprises a leaving group such as nitro, trimethylammonium, alkyl bromide, alkyl mesylate, or alkyl tosylate; or one of R^{5e} and R^{5f} is alkyne or azide, and for the remaining groups:

R^{3b} is R³ as defined above for Formula II;

R^{4b} is R⁴ as defined above for Formula II; and,

R^{5e} and R^{5f} are independently an R⁵ group as defined above for Formula II.

Examples of precursor compounds of Formula IIb are:

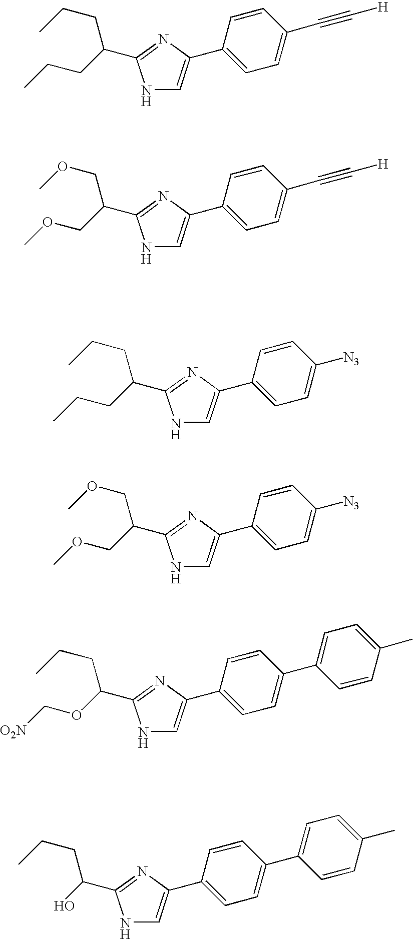

### Radiocarbonylation

Where the positron-emitting non-metal is ¹¹C, one approach to labelling with is to react a precursor compound which is the desmethylated version of a methylated compound with [¹¹C]methyl iodide. It is also possible to incorporate ¹¹C by reacting Grignard reagent of the particular hydrocarbon chain of the desired labelled compound with [¹¹C]CO₂. ¹¹C could also be introduced as a methyl group on an aromatic ring, in which case the precursor compound would include a trialkyltin group or a B(OH)₂ group.

As the half-life of ¹¹C is only 20.4 minutes, it is important that the intermediate ¹¹C moieties have high specific activity and, consequently, are produced using a reaction process which is as rapid as possible.

A thorough review of such ¹¹C-labelling techniques may be found in Antoni et al “Aspects on the Synthesis of ¹¹C-Labelled Compounds” in Handbook of Radiopharmaceuticals, Ed. M. J. Welch and C. S. Redvanly (2003, John Wiley and Sons).

For labelled compounds of Formula I, said precursor compound for radiocarbonylation is of Formula Ic:

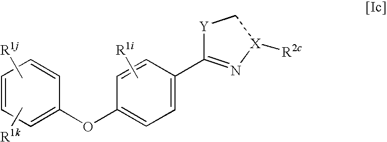

wherein:
either one of R¹ⁱ, R^{1j}, or R^{1k} is trimethyltin, tributyltin or B(OH)₂; or R^{2c} is hydrogen or hydroxyl, and for the remaining groups:
R¹ⁱ-R^{1k} are independently an R¹ group as defined above for Formula I;
R^{2c} is R² as defined above for Formula I; and,
X and Y are as defined above for Formula I.

Particular precursor compounds of Formula Ic are:

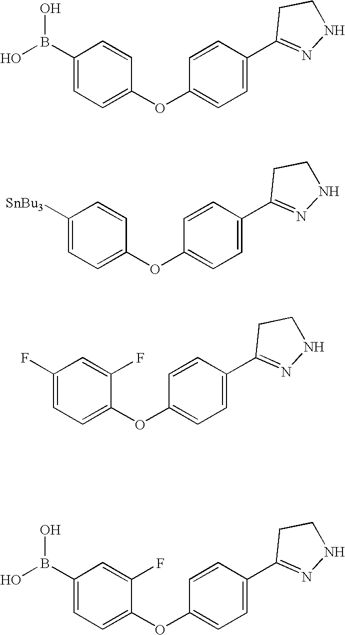

For labelled compounds of Formula II, said precursor compound for radiocarbonylation is of Formula IIc:

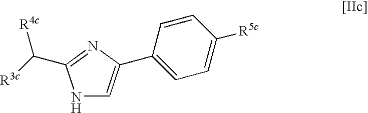

wherein either R^{5c} is hydroxyl, trimethyltin, tributyltin or B(OH)₂; or one of R³ or R^{4c} is a C₁₋₆ hydroxyalkyl, and for the remaining groups:
R^{3c} is as defined above for R³ of Formula II;
R^{4c} is as defined above for R⁴ of Formula II;
R^{5c} is as defined above for R⁵ of Formula II.

Particular precursor compounds of Formula IIc are:

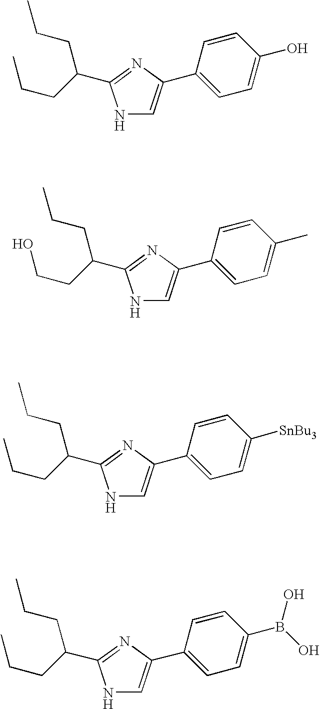

### Hyperpolarisation

By the term “hyperpolarised” is meant enhancement of the degree of polarisation of the NMR-active nucleus over its equilibrium polarisation. A number of hyperpolarisation methods are known. Certain of these are described by Golman et al [Magn. Reson. Med. 2001, 46, 1-5 and Acad. Radiol. 2002, 9 (suppl. 2), S507-S510].

The natural abundance of ¹³C (relative to ¹²C) is about 1%. Although it may be possible to carry out hyperpolarisation in a compound containing a natural abundance of the NMR active nuclei, it is preferably enriched with NNR active nuclei before administration. Suitable ¹³C-enriched compounds are suitably enriched to an abundance of at least 5%, preferably at least 50%, most preferably at least 90% before being hyperpolarised in order to obtain a labelled compound. Enrichment may include either selective enrichments of one or more sites, or uniform enrichment of all sites. This can be achieved by chemical synthesis or biological labelling.

### Radiometallation

When the detectable label is a radioactive metal ion, the labelled compound preferably comprises a metal complex of the radioactive metal ion with a synthetic ligand. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include other excipients in the preparation in vitro (e.g. radioprotectants or antimicrobial preservatives used in the preparation), or endogenous compounds in vivo (eg. glutathione, transferrin or plasma proteins). The term “synthetic” has its conventional meaning, i.e. man-made as opposed to being isolated from natural sources e.g. from the mammalian body. Such compounds have the advantage that their manufacture and impurity profile can be fully controlled.

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes, and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as MIBI (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:

(i) diaminedioximes;

(ii) N₃S ligands having a thioltriamide donor set such as MAG₃ (mercaptoacetyltriglycine) and related ligands; or having a diamidepyridinethiol donor set such as Pica;

(iii) N₂S₂ ligands having a diaminedithiol donor set such as BAT or ECD (i.e. ethylcysteinate dimer), or an amideaminedithiol donor set such as MAMA;

(iv) N₄ ligands which are open chain or macrocyclic ligands having a tetramine, amidetriamine or diamidediamine donor set, such as cyclam, monoxocyclam dioxocyclam;

(iv) N₂O₂ ligands having a diaminediphenol donor set.

Methods for the synthesis of some preferred chelating agents can be found in WO 03/006070 and WO 06/008496.

### Pharmaceutical Composition

A labelled compound of the invention is preferably administered for in vivo use in a pharmaceutical composition comprising the labelled compound, and a biocompatible carrier. A “pharmaceutical composition” is defined in the present invention as a formulation comprising a labelled compound or a salt thereof in a form suitable for administration to humans, and forms a further aspect of the invention. Administration is preferably carried out by injection of the pharmaceutical composition as an aqueous solution. Such a pharmaceutical composition may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid). Preferably, the pharmaceutical composition is a radiopharmaceutical composition, i.e. the labelled compound comprises a radioactive detectable label.

The “biocompatible carrier” is a fluid, especially a liquid, in which the labelled compound is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. For radiopharmaceutical compositions, the pre-filled syringe may optionally be provided with a syringe shield to protect the operator from radioactive dose. Suitable such radiopharmaceutical syringe shields are known in the art and preferably comprise either lead or tungsten.

The radiopharmaceutical compositions may be administered to patients for SPECT or PET imaging in amounts sufficient to yield the desired signal, typical radionuclide dosages of 0.01 to 100 mCi, preferably 0.1 to 50 mCi will normally be sufficient per 70 kg bodyweight.

The pharmaceutical compositions of the present invention may be prepared from kits. Alternatively, the pharmaceutical compositions may be prepared under aseptic manufacture conditions to give the desired sterile product. The pharmaceutical compositions may also be prepared under non-sterile conditions, followed by terminal sterilisation using e.g. gamma-irradiation, autoclaving, dry heat or chemical treatment (e.g. with ethylene oxide). Preferably, the pharmaceutical compositions of the present invention are prepared from kits. Such a kit comprises a precursor compound, preferably in sterile non-pyrogenic form, so that reaction with a sterile source of a detectable label gives the desired pharmaceutical composition with the minimum number of manipulations. Such considerations are particularly important for radiopharmaceutical compositions, in particular where the radioisotope has a relatively short half-life, and for ease of handling and hence reduced radiation dose for the radiopharmacist. Hence, the reaction medium for reconstitution of such kits is preferably a biocompatible carrier as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

Preferred aspects of the precursor compound when employed in the kit are as described above. The precursor compounds for use in the kit may be employed under aseptic manufacture conditions to give the desired sterile, non-pyrogenic material. The precursor compounds may also be employed under non-sterile conditions, followed by terminal sterilisation using e.g. gamma-irradiation, autoclaving, dry heat or chemical treatment (e.g. with ethylene oxide). Preferably, the precursor compounds are employed in sterile, non-pyrogenic form. Most preferably the sterile, non-pyrogenic precursor compounds are employed in the sealed container as described above.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. The biocompatible cation and preferred embodiments thereof are as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the imaging product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the precursor compound is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

### BRIEF DESCRIPTION OF THE EXAMPLES

Examples 1 and 2 describe synthetic routes for particular labelled compounds suitable for use in the method of the invention.

Example 3 illustrates the biodistribution of a ³H-labelled compound in a normal Wistar rat.

### EXAMPLES

### Example 1

### Synthesis of 3-[2,4-(Difluoro-phenoxy)]-phenyl-pyrazole-1-[(¹¹C)carboxylic acid] amide

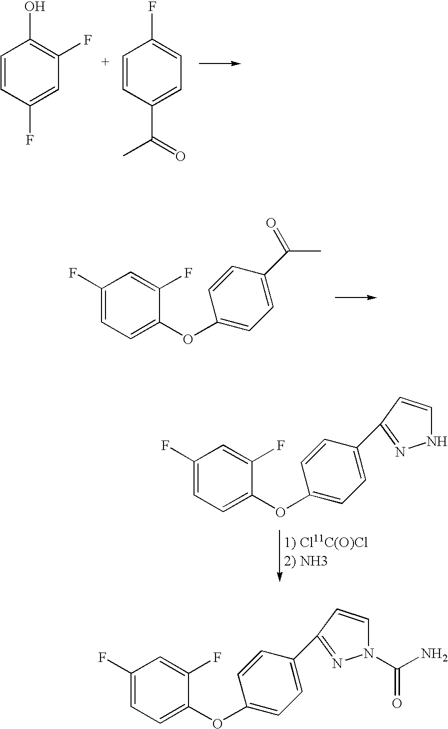

The a solution of the precursor in a suitable organic solvent (such as dichloromethane, dimethylformamide, acetonitrile or tetrahydrofuran) is introduced C-11 labelled phosgene at room temperature. Following consumption of the phosgene, ammonia is introduced (as solution or gas) and the resulting reaction mixture is heated. The crude product mixture is then purified by semi-preparative HPLC.

### Example 2

### Synthesis of 3-[2-fluoro,4-(¹⁸F-fluoro)-phenoxy]-phenyl-pyrazole-1-carboxylic acid amide

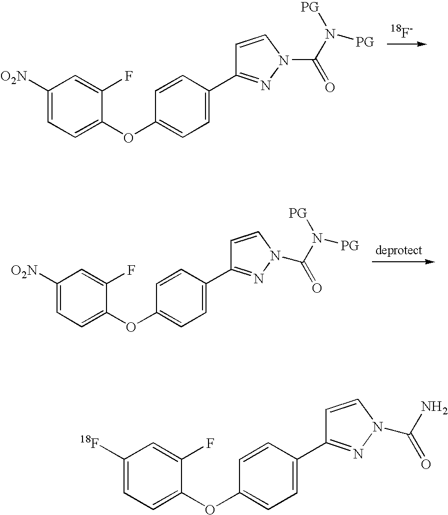

The precursor compound for ¹⁸F labelling is prepared according to the method outlined by Yang et al [J. Med. Chem. 2004 47 pp 1547-1552], with Boc protecting groups (PG in the scheme above) added to the amine prior to radiofluorination.

The precursor compound is radiofluorinated by [¹⁸F]-fluoride nucleic displacement of the aryl nitro group and subsequently deprotected by acid hydrolysis to yield the title compound.

### Example 3

### Biodistribution of ³H Compound in Rat

A tritiated version of 3-[2,4-Difluoro-phenoxy]-phenyl-pyrazole-1-carboxylic acid amide (“Hammersmith compound” in FIG. 1) was custom prepared by GE Healthcare, Cardiff.

Rats (Wistar, ca. 150 g; Charles River UK Ltd) were injected with 0.37 MBq of [³H]-3-[2,4-Difluoro-phenoxy]-phenyl-pyrazole-1-carboxylic acid amide, as an intravenous bolus via the tail vein. Rats were killed by cervical dislocation at 2, 5, 20 and 40 min post injection (p.i.). Brain (separated as cortex and hippocampus), blood, and major organs were collected, weighed and burned using a Packard Tissue Oxidizer model 307 (Packard Instrument Co., Meriden, Conn.). Samples were then counted in a β-counter (Rack Beta, Perkin Elmer LAS (UK) Ltd). The percentage of injected dose per gram (% id/g) was determined for each sample, and the results are presented in FIG. 1. This demonstrates that brain uptake of the compound was good, the time activity curves were consistent with reversible binding and the probe delineated grey and white matter (expected to have different levels of VGSCs).

What is claimed is:

1-24. (canceled)

25. A method of determining neural activity in an in vivo sample comprising detecting signals emitted by a labelled compound present in said sample, characterised in that said detecting is carried out by SPECT or PET, and wherein said labelled compound has selective affinity for the inactivated or active state of voltage-gated sodium channels.

26. The method of claim 25 which comprises the following steps:
(i) contacting the labelled compound with the sample to allow the labelled compound to bind to VGSC in the sample that are in the inactivated or active state;
(ii) detecting signals emitted by the labelled compound; and,
(iii) converting said signals into numerical data or an image.

27. The method of claim 25 wherein said labelled compound is a compound of Formula I: 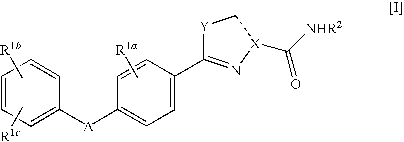
labelled with a detectable label suitable for detecting by SPECT or PET; and wherein:
R^{1a} to R^{1c} are independently an R¹ group selected from hydrogen, C₁₋₃ alkyl, C₁₋₃ alkoxy, hydroxyl, C₁₋₃ hydroxyalkyl, thiol, C₁₋₃ thioalkyl, C₁₋₃ thioalkoxy, halo, C₁₋₃ haloalkyl, C₁₋₃ haloalkoxy, nitro, C₁₋₃ nitroalkyl, C₁₋₃ nitroalkoxy, C₄₋₆ cycloalkyl, or a C₃₋₅ heterocycloalkyl group attached via a C₁₋₃ alkyl;
R² is hydrogen, C₁₋₆ alkyl, C₁₋₆ haloalkyl, or a C₄₋₆ cycloalkyl group attached via a C₁₋₆ alkyl;
A is S or O;
X is C and the dotted bond is a double bond, or X is N and the dotted bond is a single bond; and,
Y is CH₂ or CH═CH.

28. The method of claim 25 wherein said labelled compound is a compound of Formula II: 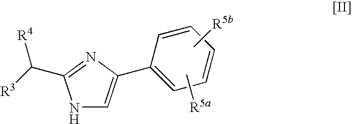
labelled with a detectable label suitable for detecting by SPECT or PET and wherein:
R³ and R⁴ are independently selected from hydrogen, C₁₋₁₀ alkyl, C₁₋₁₀ alkoxy, C₁₋₄ haloalkyl, C₁₋₄ haloalkenyl, C₁₋₃ haloalkoxy; C₄₋₆ cycloalkyl, C₁₋₂₀ PEGalkyl or C₁₋₂₀ PEG; and,
R^{5a} and R^{5b} are independently an R⁵ group selected from hydrogen, C₁₋₄ alkyl, halo, C₁₋₃ haloalkenyl, C₁₋₃ alkoxy, or is a 5- or 6-membered aromatic ring system having 0-3 heteroatoms selected from N, S and O and optionally substituted with C₁₋₃ alkyl, halo, or C₁₋₃ haloalkyl.

29. The method of claim 25 wherein said labelled compound that has selective affinity for the inactivated or active state of voltage-gated sodium channels comprises a detectable label that is either:
(i) a gamma-emitting radioactive halogen; or,
(ii) a positron-emitting radioactive non-metal.

30. A method for the diagnosis of a neurological condition that is associated with disturbed neural signalling comprising the method of claim 25.

31. The method of claim 30 wherein said in vivo sample is a human subject, or an organ or organ system within said human subject.

32. A method for monitoring the effect of treatment of a subject with a drug to combat a neurological condition associated with disturbed neural signalling comprising the step of carrying out the method of claim 25 before, during and after treatment with said drug.

33. A labelled compound of Formula I: 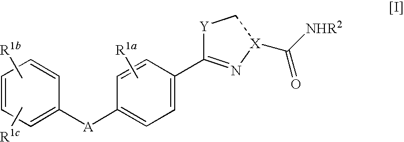
labelled with a detectable label suitable for detecting by SPECT or PET; and wherein:
R^{1a} to R^{1c} are independently an R¹ group selected from hydrogen, C₁₋₃ alkyl, C₁₋₃ alkoxy, hydroxyl, C₁₋₃ hydroxyalkyl, thiol, C₁₋₃ thioalkyl, C₁₋₃ thioalkoxy, halo, C₁₋₃ haloalkyl, C₁₋₃ haloalkoxy, nitro, C₁₋₃ nitroalkyl, C₁₋₃ nitroalkoxy, C₄₋₆ cycloalkyl, or a C₃₋₅ heterocycloalkyl group attached via a C₁₋₃ alkyl;
R² is hydrogen, C₁₋₆ alkyl, C₁₋₆ haloalkyl, or a C₄₋₆ cycloalkyl group attached via a C₁₋₆ alkyl;
A is S or O;
X is C and the dotted bond is a double bond, or X is N and the dotted bond is a single bond; and,
Y is CH₂ or CH═CH.

34. A labelled compound of Formula II: 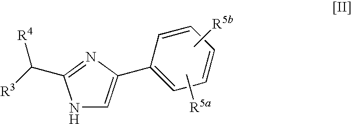
labelled with a detectable label suitable for detecting by SPECT or PET and wherein:
R³ and R⁴ are independently selected from hydrogen, C₁₋₁₀ alkyl, C₁₋₁₀ alkoxy, C₁₋₄ haloalkyl, C₁₋₄ haloalkenyl, C₁₋₃ haloalkoxy; C₄₋₆ cycloalkyl, C₁₋₂₀ PEGalkyl or C₁₋₂₀ PEG; and,
R^{5a} and R^{5b} are independently an R⁵ group selected from hydrogen, C₁₋₄ alkyl, halo, C₁₋₃ haloalkenyl, C₁₋₃ alkoxy, or is a 5- or 6-membered aromatic ring system having 0-3 heteroatoms selected from N, S and O and optionally substituted with C₁₋₃ alkyl, halo, or C₁₋₃ haloalkyl.

35. A radiopharmaceutical composition comprising a labelled compound of claim 33 and a biocompatible carrier.

36. A radiopharmaceutical composition comprising a labelled compound of claim 34 and a biocompatible carrier.
